(19) United States
(12) Patent Application Publication
(10) Pub. No.: US 2020/0147359 A1
(43) Pub. Date: May 14, 2020

(54) Microneedle Array

(71) Applicant: Esthetic Education LLC, Scottsdale, AZ (US)

(72) Inventors: Kristin Groop, Scottsdale, AZ (US); Lawrence Groop, Scottsdale, AZ (US)

(73) Assignee: Esthetic Education LLC, Scottsdale, AZ (US)

(21) Appl. No.: 16/186,576

(22) Filed: Nov. 11, 2018

(51) Int. Cl. A61M 37/00; A61B 17/34

(52) U.S. Cl. CPC A61M 37/0015; A61M 2037/0023; A61M 2037/0046; A61B 17/3417

(57) ABSTRACT

A microneedle array is provided comprising a repeating pattern of different length microneedles.

### CROSS REFERENCE TO RELATED APPLICATIONS

None

### TECHNICAL FIELD OF THE INVENTION

The present device components relate in general to dermatological and esthetic skin treatments using microneedling.

### BACKGROUND OF THE INVENTION

In dermatological and esthetic skin treatments there are at least four generalized groups of conditions that are currently being treated by handheld devices in which microneedle arrays are mounted to and utilized in the skin. These four general areas consist of age management and the treatment of fine lines and wrinkles, the treatment of scars, the treatment of dyschromias of the skin, and regenerative treatments using blood or amnion derived substances to restore areas of affected skin and hair.

There are three primary mechanisms of action involved in the use of microneedling devices in dermatology and esthetic skin treatments. The first mechanism of action involves the creation of microchannels in the skin. As the skin is an efficient barrier to the outside environment, research has shown that molecules larger than 500 Daltons cannot easily pass through the epidermis. Dermatological advances have created or identified molecules that exceed 500 Daltons that, if it were possible to bypass the epidermis, would provide some physiological benefit to the skin were they introduced into the dermis. While a hypodermic syringe achieves this, a multi-needle array of solid core needles is much more efficient. In esthetic skin procedures such as age management and the treatment of fine lines and wrinkles, while it is necessary to create microchannels to deliver large topical molecules with purported benefits to the healing processes occurring in the dermis, it is not advantageous to the healing process to inflict more damage than is necessary to create the microchannels.

The second mechanism of action involved in the use of microneedling devices in dermatology and esthetic skin treatments involves the simultaneous creation of microinjuries whilst creating microchannels. Research has shown that inflicting microinjuries to specific physiological structures induces potentially beneficial effects in the skin. The infliction of microinjuries relies on the principle of fractional injuries. As each microneedle in the array is arranged a certain distance apart from another microneedle in the array, there exists a pattern of microneedles as well as a pattern of uninjured skin as the array is introduced into the skin. Research has shown that the uninjured areas surrounding the microchannels/microinjuries act as reservoirs for healing. In using a handheld device with an array of reciprocating microneedles where the clinician can control the depth of penetration, the clinician may target a specific target or skin layer and also control the amount of microchannels and simultaneous microinjuries inflicted by varying the amount of time the device is being applied to the skin, the speed of the motor reciprocating the array, the depth of penetration selected, and the amount of “passes” across a given section of skin in the treatment area.

The third mechanism of action involved in the use of microneedling devices in dermatology and esthetic skin treatments involves the mechanical breakdown of fibrous tissue by the reciprocating microneedle array. Relatively large, dense areas of tissue can be repeatedly perforated and mechanically cut into smaller pieces by an array of sharp microneedles. This results in the angiogenesis where a blood supply is introduced into an area of avascular fibrous tissues (such as scar tissue) while simultaneously allowing collagenase to act upon (break down) irregular formations of collagen. In effect, the microneedle array “chops up” the scar tissue into smaller pieces and allows blood vessels to infiltrate the mass of scar tissue.

Research has shown that for age management and the treatment of fine lines and wrinkles, the appropriate target cell is the keratinocyte located in the lowermost layer of the epidermis. Practitioners rely on both mechanisms of action, the creation of microchannels and the creation of microinjuries. By injuring keratinocytes in the basal layer of the epidermis, the keratinocyte reacts by creating more channels between other keratinocytes. It is not necessary nor is it beneficial to penetrate into the dermis and create pinpoint bleeding if the intention is to provide age management treatments. Thus, for most patients, it is not necessary to penetrate past a depth of 1.5 mm. The clinical endpoint that is desired is moderate, uniform erythema (redness) with little to no pin-point bleeding (petechiae).

Based on surveys taken, age management treatments comprise 60% or more of all dermatological treatments performed under the classification of aesthetic services. Microneedling arrays designed for age management treatments are usually constructed with 13 or fewer needles arranged with a relatively large distance between each needle in the array. This design attempts to provide ample reservoirs of unaffected tissue to help quickly heal the areas where microchannels and relatively minor microinjuries are being created.

The treatment of scars, dyschromias and regenerative treatments for affected skin and hair require a different approach and protocol. Instead of targeting the keratinocytes as done in age management treatments, the clinician is attempting to create microchannels and microinjuries into the dermis. By creating pinpoint bleeding into the dermis, these relatively deeper microinjuries invoke the body's wound healing mechanism. This process targets the fibroblast in an effort to remodel existing collagen and create new collagen. The clinical endpoint that is desired is generalized petechiae or pin-point bleeding with the actual visible scar itself exhibiting more significant bleeding than petechiae. While there are different protocols for each of the three generalized groups of treatments, all of them require, at least, petechiae or generalized pinpoint bleeding. For treatment of scars, practitioners rely on the third mechanism of action, the mechanical breaking down of dense tissue, which requires a dense configuration of needles to efficiently and effectively achieve.

Microneedle arrays presently used in the above treatments fall generally into two basic arrangements. The first is an array with approximately 12 needles spaced relatively far apart so as to create microchannels but minimize the amount of associated microinjuries by virtue of larger reservoir areas of unaffected tissue (of course it is possible to create a more aggressive treatment using this array by administering a longer treatment with more passes over the treatment area). As stated, research has shown that erythema (redness) is the preferred clinical endpoint and this usually requires going no deeper than 1.5 mm into the skin (skin thicknesses on the face vary from as little as 0.1 mm on the forehead to 1.5 mm in the cheek region to achieve erythema but not petechiae).

One of the limitations experienced when using this pattern of microneedle arrays is that as the depth of penetration begins to exceed 2.0 mm, in many devices this array begins to exhibit “snagging” where the spring incorporated into the cartridge becomes increasingly unable to retract the array as it is inserted into the skin past 2.0 mm. This limitation has caused several manufacturers to limit the overall depth of penetration to 2.0 mm. Unfortunately, in order to effectively treat some patients with dense scars, it is necessary to penetrate the skin deeper than 2.0 mm rendering ineffective devices that cannot do so.

The second basic arrangement is an array with relatively more needles than the array described above, spaced closer together. Usually, this denser configuration results in approximately 36 needles occupying the same space on the array as the 13 needles in the prior array. These 36 needles, being closer together, are more efficient and effective at penetrating into scar tissue and creating more substantial microinjuries that are necessary for the effective treatment of scars or dyschromias, and regenerative processes, but would be too aggressive if used for age management treatments. This arrangement is ideally suited to mechanically breaking down dense scar tissue using the relatively close arrangement of microneedles that also provides more microneedles.

To meet these divergent needs, manufacturers have created two basic cartridges for sale that embody the two configurations. Practitioners choose which cartridge array design they will use based on the type of treatment being performed. In the event that a patient is being treated for both age management and another category of treatment, which is exceptionally common as most patients desire age management regardless of any other condition present, practitioners must choose to: 1) use a cartridge designed for age management and then spend extra time with extra passes to treat the other condition less effectively; 2) use a cartridge designed for scar revision and inflict unnecessary microinjury to the age management only treatment areas; or 3) use two cartridges, one designed for age management in the age management treatment areas and a second designed for scar revision in the scar revision areas.

### SUMMARY OF THE DISCLOSURE

The present invention is a microneedle array comprising microneedles of alternating length, enabling the practitioner to treat all of the four generalized groups of conditions with one array, while also minimizing the effect of snagging past a depth of 2.0 mm.

### BRIEF DESCRIPTION OF THE DRAWINGS

To provide a more complete understanding of the present invention and features and advantages thereof, reference is made to the following description, taken in conjunction with the accompanying figures, wherein like reference numerals represent like parts, in which:

FIG. 1 is a depiction of a microneedle array according to an embodiment of the present invention.

FIG. 2 is a depiction of a circular microneedle array with needles of three different lengths.

FIG. 3 is a depiction of a microneedle array according to a different embodiment having needles of three different lengths.

FIG. 4 is a depiction of another microneedle array having needles of three different lengths.

FIG. 5 is a depiction of a microneedle array having needles of four different lengths.

FIG. 6 is a depiction of a hexagonal microneedle array having needles of three different lengths.

### DETAILED DESCRIPTION OF EXAMPLE EMBODIMENTS

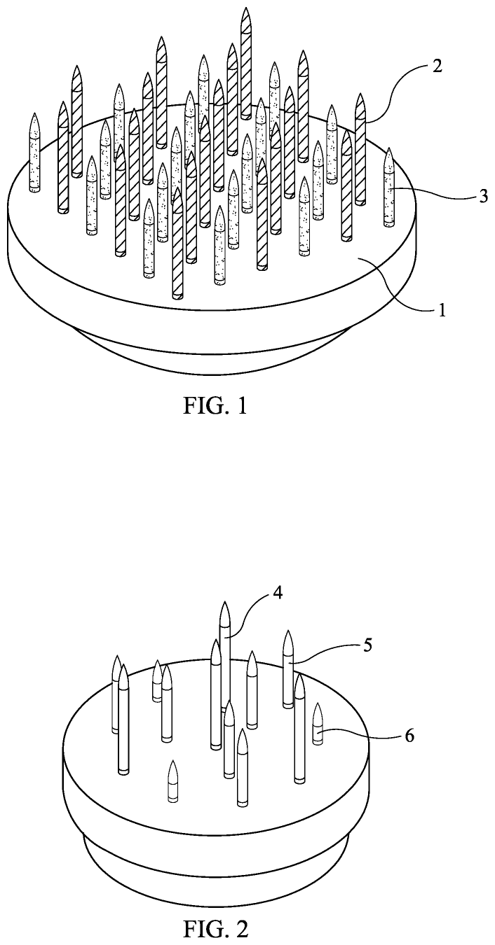

FIG. 1 illustrates an embodiment comprising a 36 pin array of microneedles for attachment to a standard microneedling device with a 1.6 mm (0.625 in) diameter circular reciprocating platform. A standard array platform 1 holds long microneedles 2 and short microneedles 3. The microneedles are either 2.5 mm or 1.0 mm, and form an alternating pattern:

| | | | | | |
|---|---|---|---|---|---|
| B | A | B | A | B | A |
| A | B | A | B | A | B |
| B | A | B | A | B | A |
| A | B | A | B | A | B |
| B | A | B | A | B | A |
| A | B | A | B | A | B |

Where A=2.5 mm and B=1.0 mm

This is an array comprising 18 spaced apart microneedles 2 that are 2.5 mm long. For the purposes of age management and some fine lines and wrinkles treatments, this configuration provides ample areas of unaffected tissue reservoirs to a depth of 1.5 mm or less, the normal maximum depth of penetration for these types of treatments.

However, if the desired treatment depth exceeds 1.5 mm, as is necessary for scar revision, dyschromias, and regenerative treatments of the hair and skin, the other 18 microneedles 3 of 1.0 mm length begin to engage.

This allows practitioners to provide the configuration best suited to all conditions by simply limiting the depth of penetration to that which research has suggested is most beneficial for a given treatment or condition. For patients receiving both age management and a treatment for one of the other types of conditions such as scar revisions, practitioners no longer have to choose between less efficient treatment of scars in favor of proper age management treatments or overtreatment of age management areas for patients receiving scar revision treatment. The disclosed configuration also makes it unnecessary to utilize two different arrays.

The configuration of the disclosed array also decreases the incidence of snagging at a penetration of 2.0 mm, as described above. The 18 microneedles 3 at 1.0 mm engage when the longer microneedles 2 reach depths past 1.5 mm, their sharp tips increasing penetration and decreasing snagging. Further, because of creation of additional microchannels that are closer together, the skin exhibits less resistance to microneedle movement as the layers and structure are disrupted.

The advantage of having two alternating lengths of needles in a uniform pattern in the array is related to the number and severity of the injury to the skin whilst creating microchannels/microinjuries. In esthetic skin procedures such as age management and the treatment of fine lines and wrinkles, while it is necessary to create microchannels to deliver large topical molecules with purported benefits to the healing processes occurring in the dermis, it is not advantageous to the healing process to inflict more damage than is necessary to create the microchannels. Increasing efficiency in both the amount of microchannels and rate at which they are created is currently accomplished by increasing the number of microneedles in the array. However, increasing the number of microneedles in the array also necessitates placing more needles closer together if one is using the same overall size array. Increasing the number of microneedles in an effort to increase efficiency in microchannel creation invariably leads to an increase in the level of injury sustained during the microchannel creation process. As the distance between microneedles decreases by virtue of adding more microneedles to a set size of array, there are more injuries created and less non-injured tissue between.

FIG. 2 shows an array comprising a 13 pin ABC circular pattern, where the microneedles have long (2.5 mm), intermediate (1.75 mm) and short (0.5 mm) lengths. The pattern of long 4, intermediate 5 and short 6 microneedles is repeated three times around the periphery of the array platform. The center of the circle contains a long 4 microneedle surrounded by three intermediate 5 microneedles. This pattern allows a greater distance between needles as the circular array shape matches the circular disk making more efficient use of the space. Reducing the number of microneedles in the array and increasing the spacing of the microneedles decreases the total amount of microinjury by virtue of fewer channels and decreases the amount of resistance as there is less total surface area of microneedles because there are fewer needles. The use of three different needle lengths allows only 4 microneedles to penetrate up to depths of 0.75 mm. This provides the clinician the option of delivering a very mild treatment for use in age management treatments. In the event there are a few areas that require a deeper penetration, 6 more needles are active from 0.75 to 2.0 mm. This provides the operator the ability to treat mild atrophic scars with minimum snagging and without overtreatment. In the event that there is a small hypertrophic scar in the area being treated for age management, 3 more needles engage from 2.0 mm to 2.5 mm.

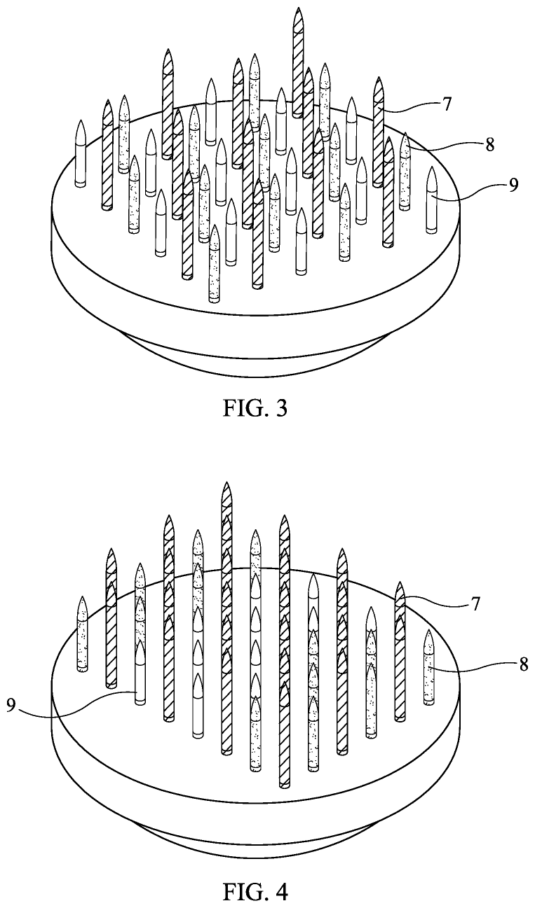

FIG. 3 shows an array holding 36 microneedles in a square pattern, with the needles in three lengths 7, 8 and 9. These may be 2.5 mm, 1.75 mm and 0.5 mm or another combination. This array offers similar advantages to the first embodiment above, but adds an intermediate length needle. Including needles of three different lengths in a square array allows the clinician to have more options in the number of microchannels and extent of microinjury. Additionally, by proportionately reducing the number of 2.5 mm needles, the total resistance of the skin to entry and withdrawal is reduced. This allows the array to be used with a smaller, less powerful motor assembly.

FIG. 4 shows a 48 pin rectangular pattern with three microneedle lengths, with A the longest 7, B intermediate 8 and C shortest 9 in the following pattern:

| | | | | | | | |
|---|---|---|---|---|---|---|---|
| A | B | A | C | A | C | A | B |
| B | A | C | A | C | A | B | A |
| A | B | A | C | A | C | A | B |
| B | A | C | A | C | A | B | A |
| A | B | A | C | A | C | A | B |
| B | A | C | A | C | A | B | A |

This array offers the same advantages as the embodiment of FIG. 3, but provides a larger array that allows the operator to increase efficiency and reduce treatment time by increasing the number of microchannels and microinjury. This array requires a relatively more powerful motor assembly, but the array will still fit on an array platform that will work with most handpieces.

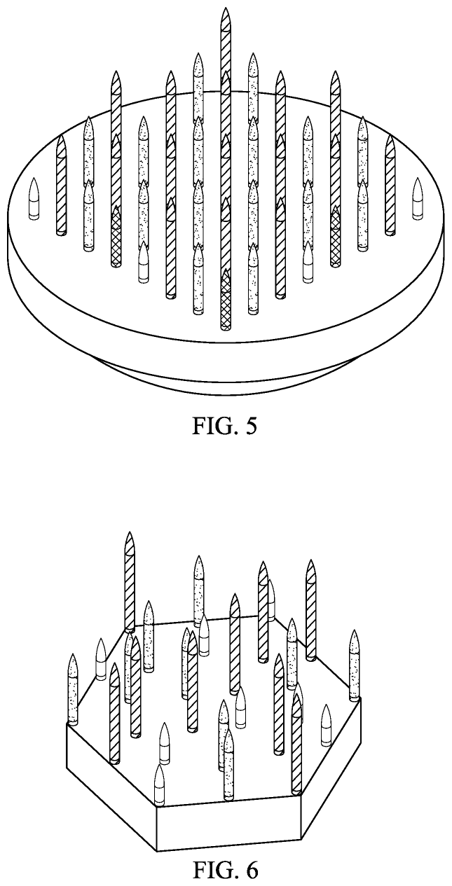

FIG. 5 shows a 64 pin square pattern with four needle lengths. This array is relatively large and provides the operator maximum depth options with maximum efficiency. This array would be ideally suited for treating large treatment areas such as the back or stomach. By utilizing needles of four different lengths in the square array, the clinician can modulate the amount of damage by changing the depth to engage more and more needles. This array would require a relatively larger array disk than the other assemblies but resistance is minimized by virtue of reducing surface area with needles of shorter lengths. A 64 pin array with all of its needles at 2.5 mm requires a significantly more powerful motor than does this array with four different needle lengths.

FIG. 6 shows a 26 pin hexagonal pattern with three different needle lengths. This embodiment incorporates the use of a hexagonal platform as well as a hexagonal array. By matching the array shape to the platform shape, the amount of space between needles can be increased while utilizing more needles than if the array were square. As with the other embodiments, the three different lengths of needles provides the clinician the ability to customize the relative amount of microinjuries by changing the depth, which allows more needles to engage. Additionally, the use of differing lengths of needles provides all the embodiments the benefit of reduced resistance to needle penetration and withdrawal.

The foregoing description has been presented and is intended for the purposes of illustration and description. It is not intended to be exhaustive nor limit the invention to the above teachings. The embodiments were chosen and described in order to best explain the principles of the invention and its practical application and to enable others skilled in the art to best utilize the invention in various embodiments and with various modifications as are suited to the particular use contemplated. Numerous other changes, substitutions, variations, alterations, and modifications may be ascertained to one skilled in the art. For example, the relative lengths of microneedles used together may be any length from a maximum of about 4 mm to a minimum of 0.1 mm, depending on the specific treatment and skin conditions. Likewise, the needle gauges employed may be varied within the range generally applicable to skin treatment. Therefore, it is intended that the invention not be limited to the particular embodiments disclosed for carrying out the invention.

What is claimed is:

1. A microneedle array for use with a microneedling instrument comprising a plurality of microneedles of at least two different microneedle lengths attached to a face of a platform, wherein adjacent microneedles are of different lengths in a pattern repeated across the face of the platform.

2. The array of claim 1 comprising microneedles of first and second lengths arranged in a plurality of rows wherein microneedles of the first length and the second length are alternated across each row.

3. The array of claim 2 comprising six rows of six microneedles wherein the first length is greater than the second length.

4. The array of claim 3 wherein the first length is approximately 2.5 mm and the second length is approximately 1.0 mm.

5. The array of claim 1 comprising microneedles of first, second and third lengths arranged in a plurality of rows wherein a pattern of microneedles of the first length, the second length and the third length is repeated across each row.

6. The array of claim 5 wherein the first length is longer than the second length and the third length is shorter than the second length.

7. The array of claim 6 comprising six rows of six microneedles, wherein the first length is about 2.5 mm, the second length is about 1.75 mm and the third length is about 0.5 mm.

8. The array of claim 1 comprising six rows of eight microneedles of first, second and third lengths, wherein patterns of pairs of lengths are repeated across each row.

9. The array of claim 1 comprising microneedles of first, second, third and fourth lengths arranged in a plurality of rows wherein a pattern of microneedles of the first length, the second length, the third length and the fourth length is repeated across each row.

10. The array of claim 9 wherein the first length is longer than the second length, the second length is longer than the third length, and the third length is longer than the fourth length.

11. The array of claim 10 comprising eight rows of eight microneedles wherein the first length is 2.5 mm, the second length is 1.75 mm, the third length is 1.25 mm and the fourth length is 0.5 mm.

12. The array of claim 1 wherein the microneedles are arranged in a plurality of concentric rings and adjacent microneedles are of different lengths in a pattern repeated around a circumference of each ring.

13. The array of claim 1 wherein the microneedles are arranged in a circumferential ring with adjacent microneedles having different lengths in a pattern repeated around the circumference of the ring and additional microneedles of different lengths are arranged inside the ring.

14. The array of claim 1 wherein the platform is hexagonal in shape and microneedles of different lengths are arranged in a repeated pattern in a peripheral hexagonal ring and in one or more internal hexagonal rings.
